(19) United States
(12) Patent Application Publication
(10) Pub. No.: US 2023/0149725 A1
(43) Pub. Date: May 18, 2023

(54) IMPLANTABLE MEDICAL DEVICES FOR COMBINATION TREATMENTS

(71) Applicant: Tigran Khachatryan, Tujunga, CA (US)

(72) Inventor: Tigran Khachatryan, Tujunga, CA (US)

(21) Appl. No.: 17/987,691

(22) Filed: Nov. 15, 2022

Related U.S. Application Data
(60) Provisional application No. 63/279,225, filed on Nov. 15, 2021.

(51) Int. Cl. A61N 1/39; A61N 1/372; A61N 1/02; A61N 1/37

(52) U.S. Cl. CPC A61N 1/3937; A61N 1/37217; A61N 1/025; A61N 1/39622; A61N 1/3706

(57) ABSTRACT

An implantable medical device configured to be planted within a subject. The medical device comprises: a first unit configured to detect pulses and output a first pulse to perform a first treatment; a second unit configured to output a second pulse to perform a second treatment; and_a control unit electrically connected to the first unit and the second unit. The control unit is configured to monitor activities of the first unit and the second unit. The control unit is also configured to prevent the first unit from mistakenly outputting the first pulses or failing to output the first pulses when a first body part of the subject needs the first pulses for the first treatment.

### BACKGROUND OF THE INVENTION

### FIELD

The disclosure relates to implantable medical devices and computer program products for treatment of cardiac arrhythmias and other cardiac dysfunctions. An implantable medical device is configured to defibrillate a patient's heart, pace the patient's heart, and/or provide baroreflex activation therapy to the patient and to avoid issues related to improper crosstalk between these functionalities.

### BACKGROUND

In a normal heart, the sinoatrial node, the heart's predominant natural pacemaker, generates electrical impulses that propagate through an electrical conduction system through the atria and then to the ventricles of the heart to excite the myocardial tissues. The atria and ventricles contract in the normal atria-ventricular sequence in synchrony as the electrical impulse propagates to result in efficient blood flow to the body to sustain life. Ventricular tachycardia (VT) and ventricular fibrillation (VF) occurs when the electrical impulses arise from the lower chambers of the heart (ventricles) and usurps control of the heart rate from the sinoatrial node. This results in desynchronized contractions between the atria and ventricles. When the heart rate reaches certain levels, the ventricles contract before they are properly filled with blood, resulting in diminished blood flow and therefore diminished oxygen throughout the body, which is life-threatening. Ventricular fibrillation (VF), in particular, stops blood flow within seconds and, if not timely and effectively treated, causes immediate death. In very few instances a heart recovers from VT/VF without treatment.

Implantable cardioverter defibrillators (ICDs) are used to treat VT, and VF. An ICD is an implantable medical device that delivers an electric shock pulse to terminate a detected tachyarrhythmia episode. The electric shock pulse depolarizes portions of the myocardium and renders it refractory. The energy of the shock pulse is provided by one or more defibrillation capacitors of the ICD.

Heart failure (HF) is a condition characterized by reduced cardiac output that triggers neurohormonal activation resulting in imbalance between sympathetic and parasympathetic activities (increased sympathetic activity, decrease in parasympathetic activity) This compensatory mechanism functions acutely to increase cardiac output and restore left ventricular (LV) functional capacity such that patients remain asymptomatic. Over time, however, sustained activation of these neurohormonal systems triggers pathologic LV remodeling and end-organ damage that ultimately drives the progression of HF.

The human body maintains blood pressure through the use of a central control mechanism located in the brain with numerous peripheral blood pressure sensing components. These components are generally made of specialized cells embedded in the walls of blood vessels that create action potentials at an increased rate as the cell is stretched. These groups of cells are generally referred to as baroreceptors. The action potentials are propagated back to the central control center via neural pathways along afferent nerves. While there are many baroreceptor components located throughout the body, there are several that are particularly important, including a baroreceptor region is located near the bifurcation of the common carotid artery into the internal and external carotid. In this area there is a small enlargement of the vessel tissues, referred to as the carotid bulb or carotid sinus. The carotid baroreceptors are generally found throughout this area. The carotid baroreceptors and related neural pathways form the primary pressure sensing component that provides signals to the brain for regulating cranial and systemic blood pressure.

Targeted stimulation of baroreceptors in the carotid sinus of HF patients can lead to decreases in sympathetic tone, peripheral vascular resistance, afterload, and heart rate. Such stimulation can be used to control blood pressure and heart rate, which are important components in the treatment of HF.

The internal jugular vein, vagus nerve, and common carotid artery (which includes the carotid sinus) are located within the carotid sheath, a fascial compartment within the neck. The carotid sheath provides relatively fixed geometric relationships between these structures while also giving some degree of insulation from surrounding tissue.

Patients with HF who have a left ventricular ejection fraction of less than or equal to 35% qualify for both ICD implantation and baroreflex activation therapy These functionalities may be provided by different systems (ICD, carotid sinus stimulator) separately implanted into the patient. However, since the baroreflex activation therapy delivers electrical impulses to the body, there is a risk that these electrical impulses may be detected by the ICD and mischaracterized as cardiac arrhythmias, and a defibrillation impulse improperly prepared and delivered to the heart. Such an improper defibrillation of the heart may result in serious morbidity or mortality. Furthermore, such improper sensing of electrical impulses from an isolated baroreflex stimulator in a patient that needs cardiac pacing could be interpreted as native cardiac signals resulting in inhibition of pacing. This would again result in significant morbidity or mortality.

Accordingly, there is a need for implantable medical devices and systems capable of providing cardiac pacing, arrhythmia detection and defibrillation, and baroreflex activation therapy to a patient in a single unit to mitigate the above-mentioned risks and negate the need for multiple pulse generator implantations. The present invention addresses this unmet need.

### SUMMARY

In general, in the absence of one or more aspects of the present disclosure, if an ICD and/or a pacemaker were to be combined with baroreflex activation therapy in a patient, the ICD and/or the pacemaker may detect an electrical impulse from a carotid sinus stimulator lead of a baroreflex activation therapy device and erroneously interpret this impulse as a heartbeat. This misinterpretation may cause the ICD and/or the pacemaker to defibrillate the heart (ICD) and/or fail to pace the heart (pacemaker), either or both of which can cause significant morbidity and mortality. In patients receiving ICD and/or pacemaker therapy, in addition to baroreflex activation therapy, these different therapeutic modalities must be effectively coordinated so that one therapy doesn't interfere with another therapy. This and other benefits are provided by the present disclosure.

In one aspect, the disclosure provides an implantable medical device, comprising a defibrillation lead and/or a pacemaker lead, and a carotid sinus stimulator lead, and these leads are operatively connectable to a processor. The processor is configured to coordinate the defibrillation of the heart, the maintenance of the pace of the heart, and the delivery of baroreflex activation therapy, and prevent an improper defibrillation of the heart and/or an improper pacemaker activity (e.g., an inhibition of the pacemaker) that would result from a detection of an electrical impulse that originated from the delivery of baroreflex activation therapy. In implementations, the implantable medical device further comprises a non-transitory machine-readable medium having instructions stored thereon which, when executed by the processor, configure the processor in this manner.

In another aspect, the disclosure provides a computer program product comprising a non-transitory machine-readable medium having instructions stored thereon which, when executed by a processor, configure the processor to coordinate a defibrillation of a heart of a subject, a maintenance of a pace of the heart of the subject, and a delivery of baroreflex activation therapy to the subject, and prevent an improper defibrillation of the heart and/or an improper pacemaker activity (e.g., an inhibition of the pacemaker) that would result from a detection of an electrical impulse that originated from the delivery of baroreflex activation therapy. The computer program product may be provided as executable computer code stored on a suitable non-transitory computer-readable storage medium.

In various implementations, the processor is further configured to monitor an electrical activity of the heart of the subject, analyze the electrical activity of the heart of the subject for an analysis, and determine whether the heart of the subject is undergoing a cardiac arrhythmia and/or needs to be paced based on the analysis. The analysis may include analyzing a detected electrical impulse to characterize the detected electrical impulse as having originated from the delivery of baroreflex activation therapy and as not having originated from the electrical activity of the heart of the subject. The analysis may include adjusting one or more parameters associated with an electrical activity of the heart (e.g., a parameter associated with detection of a cardiac arrhythmia) such that an electrical impulse that originated from the delivery of baroreflex activation therapy is not mischaracterized as a normal heartbeat or an arrhythmia and does not lead to an improper inhibition of pacemaker activity or an improper defibrillation of the heart, respectively.

Accordingly, an object of the present disclosure is to provide improved implantable medical devices and systems for management of heart health in HF patients and avoiding improper cardiac defibrillation and improper inhibition of pacemaker activity as a result of the presence of multiple therapeutic modalities of the device or system during use. Another object of the present disclosure is to provide computer program products that may be used with or incorporated into existing or future implantable medical devices and systems for improved management of heart health.

Other objects, features and advantages of the present invention will become apparent from the following detailed description taken in conjunction with the accompanying drawings.

### BRIEF DESCRIPTION OF THE DRAWINGS

Although the characteristic features of the invention will be particularly pointed out in the claims, exemplary implementations of the invention and manners in which they may be made and used may be better understood after a review of the following description, taken in connection with the accompanying drawings, wherein like numeral annotations are provided throughout.

FIG. 1 depicts an illustration of an exemplary implantable medical device with ICD lead(s) (top) and pacemaker lead(s) (bottom) implanted into a left sub-clavicular area of a patient.

FIG. 2A depicts a schematic of the exemplary implantable medical device showing leads and components of the device.

FIG. 2B depicts another embodiment of the implantable medical device showing leads and components of the device.

FIG. 3 depicts a schematic of components of exemplary instructions of the memory of the implantable medical device.

FIG. 4A depicts a flowchart of an analysis process of an exemplary control module of the instructions.

FIG. 4B depicts a flowchart of a parameter adjustment process of an exemplary control module of the instructions.

### DETAILED DESCRIPTION OF THE INVENTION

Reference is made herein to the attached drawings. Like reference numerals may be used in the drawings to indicate like or similar elements of the description. The figures are intended for representative purposes, are not drawn to scale, and should not be considered limiting.

Unless otherwise defined herein, terms and phrases used in connection with the present disclosure shall have the meanings that are commonly understood by those of ordinary skill in the art.

As used in the description and in the claims, the terms “comprising” and “comprises” do not exclude other elements or steps. Where an indefinite or definite article is used when referring to a singular noun, e.g., “a,” “an,” or “the,” this includes a plural of that noun unless something else is specifically stated. Furthermore, the terms first, second, third, and the like in the description and in the claims, are used for distinguishing between elements and not necessarily for describing a sequential or chronological order. It is to be understood that the terms so used are interchangeable under appropriate circumstances and that the implementations of the disclosure described herein are capable of operation in other sequences than described or illustrated herein.

As used herein, the term “about” refers to the usual error range for the respective value readily known to the skilled person in this technical field. Reference to “about” a value or parameter herein includes and describes implementations that are directed to that value or parameter per se.

As used herein, the term “processor” refers to a single-core processor, a single processor with software and/or hardware multi-thread execution capability, a multi-core processor, a multi-core processor with software and/or hardware multi-thread execution capability, hardware circuitry configured to perform operations, or any computing or processing unit or computing device including, but not limited to a parallel platform, a parallel platform having distributed shared memory, an integrated circuit, an application specific integrated circuit (ASIC), a digital signal processor (DSP), a field programmable gate array (FPGA), a programmable logic controller (PLC), a complex programmable logic device (CPLD), a discrete gate logic, a transistor logic, discrete hardware components, or any combination thereof designed to perform the functions described herein. The processor may utilize a nanoscale architecture, such as molecular or quantum dot-based transistors, switches, and gates to optimize space usage or enhance the performance of a medical device or system. The processor may be implemented as a combination of computing or processing units.

As used herein, the terms “memory,” “medium,” and “storage medium” refer to any non-transitory form of computer-readable medium and/or machine-readable medium that may be used to store, among other items, instructions that are executable by one or more processors. The memory disclosed herein can include volatile memory or non-volatile memory or can include both volatile and non-volatile memory. By way of example, and not limitation, non-volatile memory may include read-only memory (ROM), programmable ROM (PROM), electrically programmable ROM (EPROM), electrically erasable PROM (EEPROM), or flash memory. Volatile memory may include random access memory (RAM), such as static RAM (SRAM), dynamic RAM (DRAM), synchronous DRAM (SDRAM), double data rate SDRAM (DDR SD RAM), enhanced SD RAM (ESDRAM), sync link DRAM (SLDRAM), or direct Rambus RAM (DRRAM). The memory is intended to include, without limitation, any of these and any other suitable type of memory.

The various exemplary logic blocks, modules, processors, means, circuits, and algorithm steps described in connection with aspects disclosed herein are electronic hardware (e.g., source code or various forms of digital implementations, analog implementations, or a combination of the two), programs or design code that incorporate instructions (which may be designed using any other technique, referred to herein as “software” for convenience). It should be further understood that software and/or instructions may be implemented as a “module” (representative of a group of functionalities) or a combination of modules (representative of a combination of groups of functionalities). The processes, methods, and operations disclosed herein may be implemented as dedicated hardware or hardware executing software. To clearly illustrate this interchangeability between hardware and software, various illustrative components, blocks, modules, circuits, and steps are disclosed generally in terms of their functionality. Whether such functionality is implemented as hardware or software depends upon the particular application and design constraints imposed on the overall system. Those skilled in the art can implement the described functionality in any of a variety of ways for each particular application, but such implementation decisions should not be construed as causing a departure from the scope of the present disclosure.

The present disclosure provides a device configured to provide cardiac defibrillation, pacemaker therapy, and baroreflex activation therapy and to minimize risk of improper defibrillation and/or improper pacemaker inhibition due to electrical impulses of the baroreflex activation therapy. The baroreflex activation therapy may involve stimulation of nervous system targets such as the vagus nerve and/or its branches, the carotid artery, the carotid sinus nerve and/or its branches, baroreceptors, and/or for otherwise activating a baroreceptor response. These therapies are useful for controlling heart rate and/or regulating blood pressure for treatment of hypertension, congestive heart failure, or other conditions.

In implementations of the baroreflex activation therapy, contents of the carotid sinus may be stimulated transvascularly or subcutaneously (e.g., by advancing and/or surgically suturing) an energy delivery element, which may be an electrode, and energizing the energy delivery element to direct energy to target contents of the carotid sinus. The energy may be directed to a carotid artery at the carotid sinus, and/or to a carotid sinus nerve or nerve branch within the carotid sinus, to nerve branches emanating from carotid artery baroreceptors, and/or to a vagus nerve or nerve branch within the carotid sinus.

In various implementations of the baroreflex activation therapy, shielding may be used to minimize collateral stimulation of unintended targets, and a shield may be positioned at least partially surrounding the carotid sinus sheath. The shield may help block conduction of energy beyond the sheath during energization of the energy delivery element.

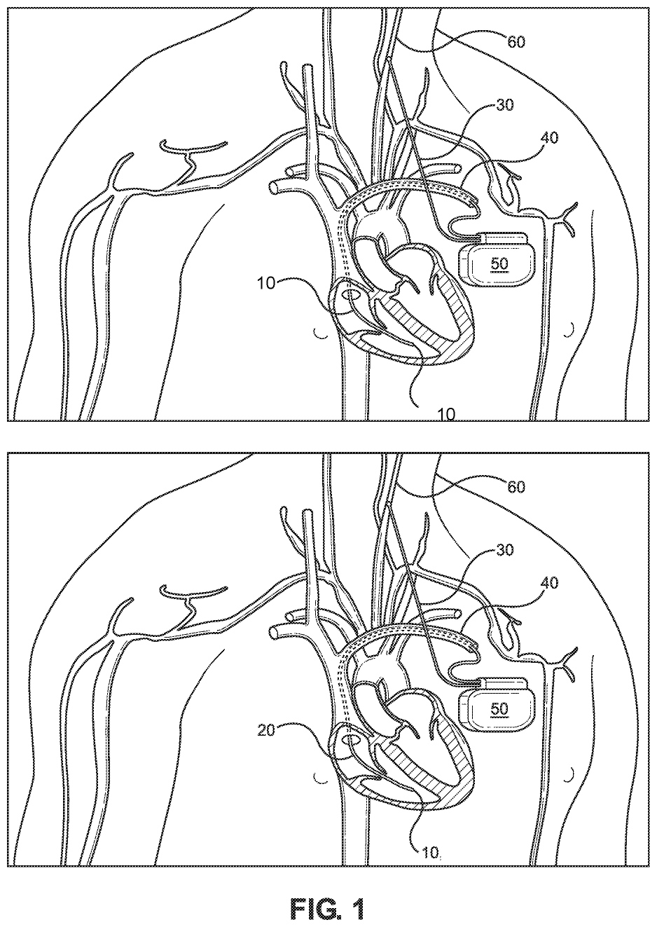

Referring now to FIG. 1, there is depicted an illustration of an exemplary implantable medical device with ICD lead(s) 10 (top) and pacemaker lead(s) 20 (bottom) implanted into a left sub-clavicular area of a patient. The implantable medical device includes a pulse generator 50 connected to various leads, such as a defibrillation lead 10 configurable for a defibrillation of a heart of a subject, a pacemaker lead 20 configurable for a maintenance of a pace of the heart of the subject, and a carotid sinus stimulator lead 30 configurable for a delivery of baroreflex activation therapy to the subject. The defibrillation lead 10 and the pacemaker lead 20 (e.g., a combination ICD-pacemaker lead and/or separate ICD and pacemaker leads, top and bottom of FIG. 1, respectively) may be transvenous and enter the heart through the left subclavian vein 40 and the tip of the ICD/Pacemaker lead 10, 20 combination may contact the right ventricular myocardium, as shown, for monitoring the heart's electrical activity, delivering pacemaker impulses, and/or delivering defibrillation impulses. The carotid sinus stimulator lead 30 may extend upward from the pulse generator 50 toward the left carotid sinus 60 (as shown) and/or the right carotid sinus for delivery of electrical impulses for the baroreflex activation therapy. The device may be inserted into the right subclavicular area or the left subclavicular area 40 (as shown).

Further, in the embodiment illustrated in FIG. 1, the implantable medical device makes use of a combination of ICD lead and Pacemaker lead, wherein the tip of the lead combination is located in the right ventricular myocardium. In one embodiment, the implantable medical device may make use of only one Pacemaker lead, wherein the tip of the Pacemaker lead is located in the atrium or ventricle chamber. In yet another embodiment, the implantable medical device may make use of a ICD lead, (whose tip is located in the ventricle chamber) and a Pacemaker lead (whose lead is located in the atrium chamber).

In certain implementations, the device may be provided with the carotid sinus stimulator lead(s) 30, with the carotid sinus stimulator lead(s) 30 combined with the ICD lead(s) 10, with the carotid sinus stimulator lead(s) 30 combined with the separate ICD and pacemaker leads 10, 20, with the carotid sinus stimulator lead(s) 30 combined with the pacemaker lead(s) 20, or with the carotid sinus stimulator lead(s) 30 combined with the combination ICD and pacemaker lead(s) 10, 20. In certain instances, a patient may qualify for cardiac pacing, baroreflex activation therapy, and monitoring for cardiac defibrillation, and these functions and their corresponding structures may be provided together in one device or system of the present disclosure.

Further, the physical configuration of the leads in a subject's body may differ in different embodiments based on the location of the pulse generator. In the embodiment illustrated in FIG. 1, the pulse generator 50 is placed in the left portion of a subject's chest, wherein ICD lead(s) 10 (top) and pacemaker lead(s) 20 (bottom) enter the heart through the left sub-clavicular vein. However, in different embodiments, the pulse generator 50 can be placed in the right portion of the subject's chest, wherein ICD lead(s) 10 (top) and pacemaker lead(s) 20 (bottom) enter the heart through the right sub-clavicular vein. Similarly, in the embodiment illustrated in FIG. 1, the carotid sinus stimulator lead 30 may extend upward from the pulse generator 50 toward the left carotid sinus. In the embodiments where the pulse generator 50 is placed in the right portion of a subject's chest, the carotid sinus stimulator lead 30 may extend upward from the pulse generator 50 toward the right carotid sinus.

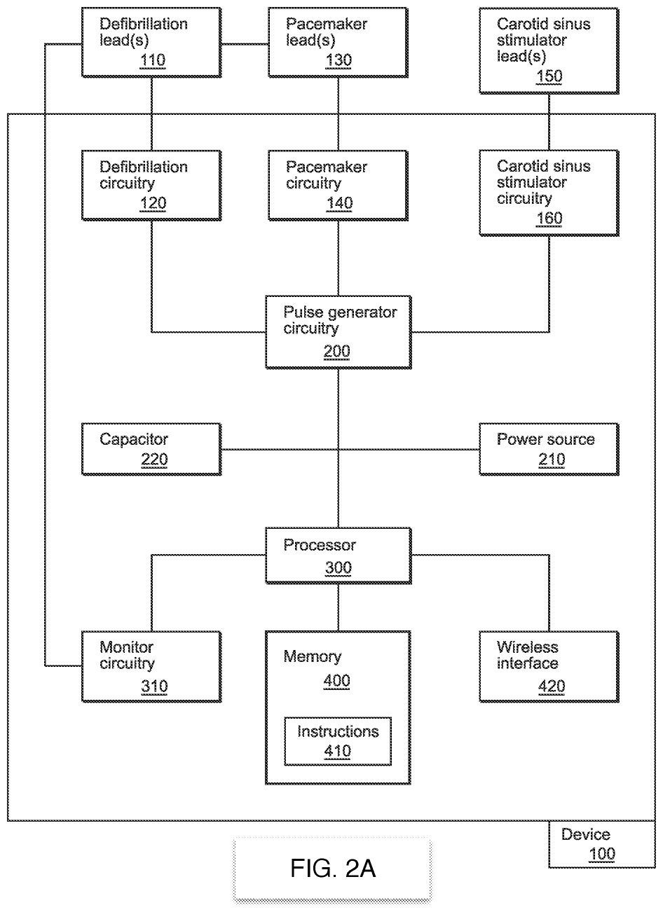

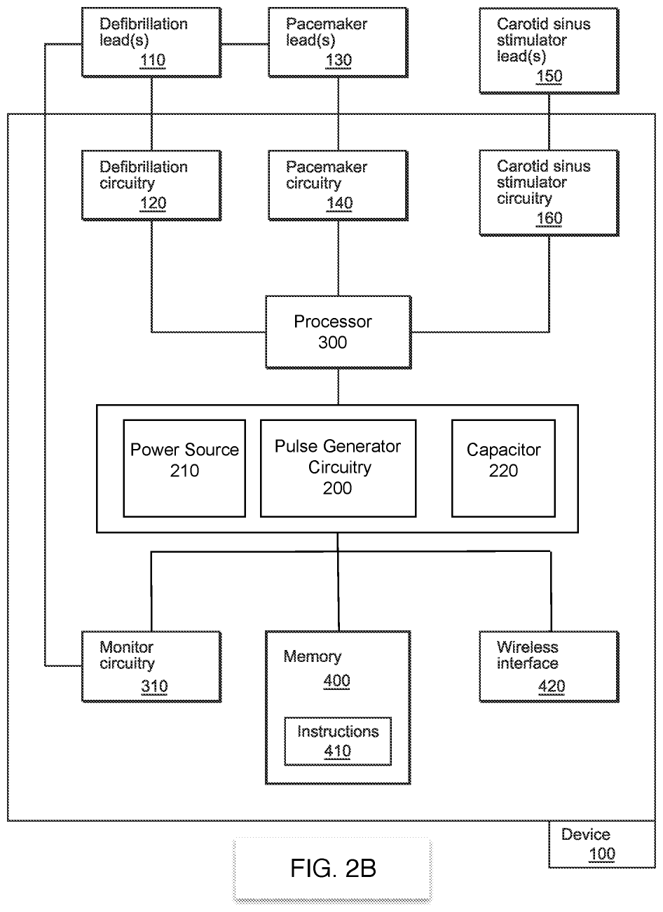

Referring now to FIG. 2A, there is depicted a schematic of the exemplary implantable medical device 100 showing leads and components of the device 100. Generally, the disclosure provides an implantable medical device 100, comprising a defibrillation lead 110 configurable for a defibrillation of a heart of a subject, a pacemaker lead 130 configurable for a maintenance of a pace of the heart of the subject, and a carotid sinus stimulator lead 150 configurable for a delivery of baroreflex activation therapy to the subject. The defibrillation lead 110, the pacemaker lead 130, and the carotid sinus stimulator lead 150 are operatively connectable to a processor 300, and the processor 300 is configured to coordinate the defibrillation of the heart, the maintenance of the pace of the heart, and the delivery of baroreflex activation therapy. The processor 300 is also configured to prevent an improper defibrillation of the heart, and/or prevent an improper inhibition of pacemaker activity, that would result from a detection of an electrical impulse that originated from the delivery of baroreflex activation therapy. In various implementations, the implantable medical device 100 includes a memory 400 (e.g., a non-transitory machine-readable medium) having instructions 410 stored thereon which, when executed by the processor 300, configure the processor 300 to perform these and/or other functionalities of the present disclosure.

In implementations, the defibrillation lead 110 is operably connectable to the processor 300 via a defibrillation circuitry 120, the pacemaker lead 130 is operably connectable to the processor 300 via a pacemaker circuitry 140, and/or the carotid sinus stimulator lead 150 is operably connectable to the processor 300 via a carotid sinus stimulator circuitry 160. The defibrillation circuitry 120, the pacemaker circuitry 140, and/or the carotid sinus stimulator circuitry 160 may be operably connected to the processor 300 via a pulse generator circuitry 200. The device 100 includes a power source 210 operably connected to a capacitor 220 configured for a discharge of an electrical impulse for the defibrillation of the heart, the maintenance of the pace of the heart, and/or the delivery of baroreflex activation therapy. A certain implementation of these components of the device 100 is shown in the figure, however, alternate implementations may be made without departing from the scope of the present disclosure.

In implementations, the device 100 includes a wireless interface 420, which may comprise a wireless transceiver, for sending and receiving data as part of a wireless communication between the implantable medical device and another device, such as a personal computer, a workstation, a mobile phone, a tablet, etc. A wireless communication protocol may be used for programming, initializing, troubleshooting, or otherwise interacting with the device 100 for maintenance, upgrades, repairs, firmware and/or software updates, etc. The wireless interface 420 may use any suitable wavelength(s) and/or wireless communication protocol(s) for sending and receiving data packets. A certain implementation of these components of the device 100 is shown in the figure, however, alternate implementations may be made without departing from the scope of the present disclosure. For instance, the wireless interface 420 may transmit data to be processed by an external device and receive processed data from said external device.

FIG. 2B is a schematic view depicting the implantable medical device according to another embodiment of the present invention. In the present embodiment, the pulse generator circuity 200 is integrated into the processor 300. Also, the capacitor 220 that stores energy is integrated into the power source 210 which directly supplies power to the processor 300. Accordingly, the process 300 of the present embodiment is responsible for analyzing pulses and generates pulses for the defibrillation lead 110, pacemaker lead 130, and carotid sinus stimulator lead 150 to perform treatments.

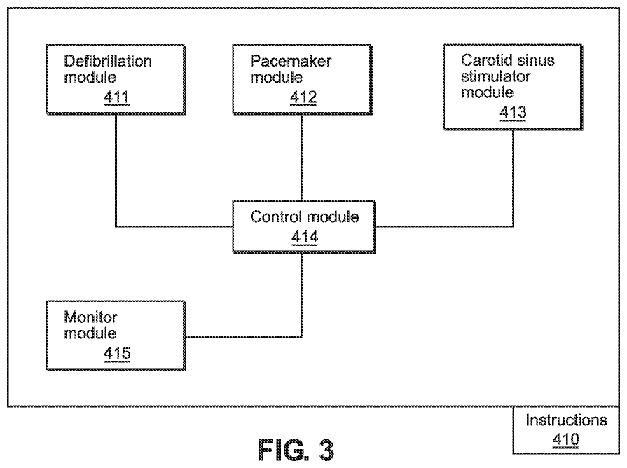

Referring now to FIG. 3, there is depicted a schematic of components of exemplary instructions 410 of the memory 400 of the implantable medical device 100. The instructions 410 of the memory 400 may include executable software and/or firmware in the form of one or more modules, e.g., a defibrillation module 411, a pacemaker module 412, a carotid sinus stimulator module 413, a monitor module 414, and a control module 415. While shown as these several modules, the functionalities embodied may be present as fewer or greater numbers of modules or other units of executable code according to a particular implementation. The defibrillation module 411, the pacemaker module 412, and the carotid sinus stimulator module 413 may receive data, process data, and transmit results for initiating, sustaining, and/or terminating the delivery of electrical impulses from the device 100 to the patient as part of defibrillating the heart, maintaining pace of the heart, and/or delivering baroreflex activation therapy to the patient, respectively. The control module 414 may receive data, process data, and transmit results for coordinating activities of the defibrillation module 411, the pacemaker module 412, and the carotid sinus stimulator module 413 accounting for data received from the monitor module 415. The monitor module 415 may receive data (e.g., data related to an electrical activity of the heart of the subject), process data, and transmit results (e.g., to the control module) for keeping the control module 414 informed about the status of the heart. A certain implementation of these components of the instructions is shown in the figure, however, alternate implementations may be made without departing from the scope of the present disclosure.

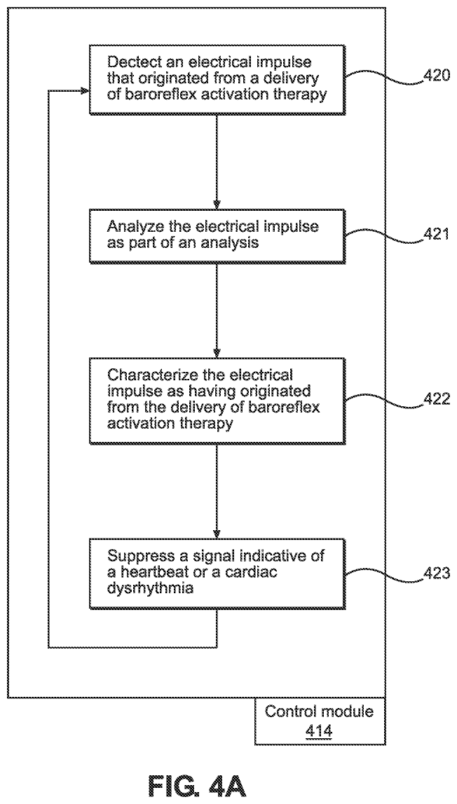

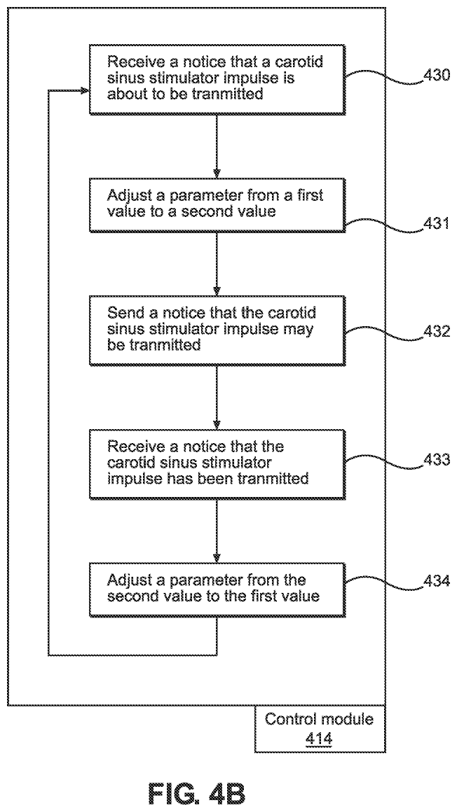

Referring now to FIG. 4A, there is depicted a flowchart of an analysis process of an exemplary control module 414 of the instructions. The control module 414 (and/or another module, such as the monitor module 415) may include step 420 of configuring the processor 300 to detect an electrical impulse that originated from a delivery of baroreflex activation therapy, step 421 of analyzing the electrical impulse as part of an analysis, step 422 of characterizing the electrical impulse as having originated from the delivery of baroreflex activation therapy, and step 423 of suppressing (or otherwise deactivate or ignore) a signal indicative of a heartbeat or a cardiac dysrhythmia. In this manner, the control module 414 (and/or another module, such as the monitor module) may prevent inhibition of a pacemaker activity and/or may prevent initiation of a defibrillation process that would result in lack of needed pacemaker function and introduction of an improper defibrillation of the heart, respectively, due to a false positive detection of a heartbeat. The analysis may include analyzing timing of detected impulses as well as analyzing other characteristics of detected impulses, such as intensity and duration, to determine whether the detected impulses originated from baroreflex activation therapy or from the patient's heart.

In implementations, the defibrillation lead 110 and/or the pacemaker lead 130 is connected to the processor 300 via a monitor circuitry 310 for transmitting signals that relate to an electrical activity of the heart of the subject to an input of the processor 300 for analysis. Accordingly, the processor 300 may be further configured to monitor the electrical activity of the heart of the subject, analyze the electrical activity for an analysis, and determine whether the heart of the subject is undergoing a cardiac arrhythmia and/or needs to be paced based on the analysis. The processor 300 may be further configured to detect the electrical impulse that originated from the delivery of baroreflex activation therapy, analyze the electrical impulse as part of the analysis, and characterize the electrical impulse as having originated from the delivery of baroreflex activation therapy and as not having originated from the electrical activity of the heart of the subject.

Referring now to FIG. 4B, there is depicted a flowchart of a parameter adjustment process of an exemplary control module 414 of the instructions. A parameter adjustment process (e.g., of the control module 414) may work in cooperation with a carotid sinus stimulation process (e.g., of the carotid sinus stimulator module) to coordinate the interpretation of electrical impulses with the carotid sinus stimulation process to avoid false positive detection of cardiac arrhythmias. The parameter adjustment process includes step 430 that may be carried out by a processor 300 configured to receive a notice that a carotid sinus stimulator impulse is about to be transmitted, step 431 of adjusting a parameter associated with detection of a cardiac arrhythmia (e.g., a parameter managed by the control module and/or the monitor module) from a first value to a second value, and step 432 of sending a notice that the carotid sinus stimulator impulse may be transmitted. After the carotid sinus stimulator impulse is transmitted, the parameter adjustment process may include step 433 in which the processor 300 may receive a notice that the carotid sinus stimulator impulse has been transmitted and step 434 of adjusting the parameter from the second value to the first value. The first value of the parameter may be suitable for detecting bona fide cardiac rhythms and/or arrhythmias, and the second value of the parameter may be suitable for suppressing signals related to a false positive detection of a heartbeat and/or a cardiac arrythmia that is actually an electrical impulse that originated from the carotid sinus stimulator lead.

Accordingly, in implementations, the processor 300 may be further configured to adjust a parameter associated with the electrical activity of the heart of the subject from a first value to a second value, such that the first value is associated with generation of a signal indicative of the heartbeat and/or the cardiac arrhythmia, and the second value is not associated with generation of the signal indicative of the heartbeat and/or the cardiac arrhythmia. The processor 300 may be further configured to adjust the parameter associated with the electrical activity of the heart of the subject from the second value to the first value to return the processor to a monitoring state.

The foregoing descriptions of specific implementations have been presented for purposes of illustration and description. They are not intended to be exhaustive or to limit the invention to the precise forms disclosed, and modifications and variations are possible in view of the above teaching. The exemplary implementations were chosen and described to best explain the principles of the invention and its practical application, to thereby enable others skilled in the art to best utilize the invention and its implementations with modifications as suited to the use contemplated.

It is therefore submitted that the invention has been shown and described in the most practical and exemplary implementations. It should be recognized that departures may be made which fall within the scope of the invention. With respect to the description provided herein, it is submitted that the optimal features of the invention include variations in size, materials, shape, form, function, manner of operation, assembly, and use. All structures, functions, and relationships equivalent or essentially equivalent to those disclosed are intended to be encompassed by the invention.

1. An implantable medical device configured to be planted within a subject, the medical device comprising:
a first unit configured to detect pulses and output a first pulse to perform a first treatment;
a second unit configured to output a second pulse to perform a second treatment; and
a control unit electrically connected to the first unit and the second unit, wherein the control unit is configured to:
monitor pulses and activities of the first unit and the second unit; and
prevent the first unit from mistakenly outputting the first pulses or failing to output the first pulses when a first body part of the subject needs the first pulses for the first treatment.

2. The implantable medical device of claim 1, wherein the control unit includes:
a processor; and
a monitor unit connected to the processor, the monitor unit being configured to monitor pulses and generate monitor results based on the pulses detected,
wherein the processor analyzes the monitor results and determines whether the detected pulses originates from the second unit or from the first body part in need of the first pulses for the first treatment, the processor notifies the first unit to not generate the first pulses if the detected pulses are determined to originate from the second unit.

3. The implantable medical device of claim 2, wherein the control unit includes:
a first parameter associated with pulses generated by the first body part in need of the first pulses for the first treatment; and
a second parameter associated with the second pulses originating from the second unit; wherein
the second unit notifies the control unit before outputting the second pulses, the processor then analyzes the monitor results based on the second parameter; wherein
the processor analyzes the monitor results based on the first parameter after the second unit has finished outputting the second pulses.

4. The implantable medical device of claim 1, wherein the control unit includes:
a processor; and
a monitor unit connected to the processor, the monitor unit being configured to monitor pulses and generate monitor results based on the pulses detected,
wherein the processor analyzes the monitor results and determines whether the pulses detected originates from the second unit or from the functioning first body part, the processor notifies the first unit if the pulses detected are determined to originate from the second unit.

5. The implantable medical device of claim 4, wherein the control unit includes a second parameter associated with the second pulses originating from the second unit; the second unit notifies the control unit before outputting the second pulses, the processor then analyzes the monitor results based on the second parameter.

6. The implantable medical device of claim 1, wherein the second unit notifies the control unit before outputting the second pulses, the control unit notifies the first unit before the second units outputs the second pulses, the first unit then monitors pulses based on a second parameter associated with the second pulses from the second unit.

7. The implantable medical device of claim 1, wherein the control unit monitors characteristics of pulses and generates monitor result that includes the characteristics.

8. The implantable medical device of claim 1, further comprising a wireless unit electrically connected to the control unit, the wireless unit being configured to receive first data from the control unit and wirelessly output the first data to an external device, the wireless unit being configured to wirelessly receive second data from the external device and transfer the second data to the control unit.

9. The implantable medical device of claim 1, further comprising a wireless unit electrically connected to the control unit, the wireless unit being configured to wirelessly receive update data from an external device, the wireless unit transferring the update data to the control unit for performing parameter update or firmware update on at least one of the first unit, the second unit, the control unit, and the wireless unit.

10. A method of using an implantable medical device to provide multiple medical treatments, comprising the steps of:
implanting a medical device in a body of a subject, wherein the medical device includes:
a first unit configured to detect pulses and output a first pulse to perform a first treatment;
a second unit configured to output a second pulse to perform a second treatment; and
a control unit electrically connected to the first unit and the second unit;
configuring the control unit to monitor pulses and activities of the first unit and the second unit; and
configuring the control unit to prevent the first unit from mistakenly outputting the first pulses or failing to output the first pulses when a first body part of the subject needs the first pulses for the first treatment.

11. The method of using the implantable medical device of claim 10, wherein the control unit includes
a processor and a monitor unit, the method comprises:
connecting the processor to the monitor unit;
configuring the monitor unit to monitor pulses and generate monitor results based on the pulses detected;
configuring the processor to analyze the monitor results and determines whether the detected pulses originates from the second unit or from the first body part in need of the first pulses for the first treatment; and
configuring the processor to notifies the first unit to not generate the first pulses if the detected pulses are determined to originate from the second unit.

12. The method of using the implantable medical device of claim 11, further comprising:
configuring the control unit to include:
a first parameter associated with pulses generated by the first body part in need of the first pulses for the first treatment; and
a second parameter associated with the second pulses originating from the second unit;
configuring the second unit to notify the control unit before outputting the second pulses and the processor to analyzes the monitor results based on the second parameter; and
configuring the processor to analyze the monitor results based on the first parameter after the second unit has finished outputting the second pulses.

13. The method of using the implantable medical device of claim 10, wherein the control unit includes
a processor and a monitor unit, the method comprises:
connecting the monitor unit to the processor;
configuring the monitor unit to monitor pulses and generate monitor results based on the pulses detected;
configuring the processor to analyze the monitor results and determine whether the pulses detected originates from the second unit or from the functioning first body part; and
configuring the processor to notify the first unit if the detected pulses are determined to originate from the second unit.

14. The method of using the implantable medical device of claim 13, further comprising:
configuring the control unit to include a second parameter associated with the second pulses;
configuring the second unit to notify the control unit before outputting the second pulses; and
configuring the processor to analyze the monitor results based on the second parameter.

15. The method of using the implantable medical device of claim 10, further comprising:
configuring the second unit to notify the control unit before outputting the second pulses;
configuring the control unit to notify the first unit before the second units outputs the second pulses; and
configuring the first unit monitors pulses based on a second parameter associated with the second pulses from the second unit.

16. The method of using the implantable medical device of claim 10, further comprising configuring the control unit to monitor characteristics of pulses and generate monitor result that includes the characteristics.

17. The method of using the implantable medical device of claim 10, further comprising:
connecting a wireless unit to the control unit;
configuring the wireless unit to receive first data from the control unit and wirelessly output the first data to an external device; and
configuring the wireless unit to wirelessly receive second data from the external device and transfer the second data to the control unit.

18. The method of using the implantable medical device of claim 10, further comprising:
connecting a wireless unit to the control unit;
configuring the wireless unit to wirelessly receive update data from an external device; and
configuring the wireless unit to transfer the update data to the control unit for performing parameter update or firmware update on at least one of the first unit, the second unit, the control unit, and the wireless unit.

19. An implantable medical device in a body of a subject, the medical device comprising:
a first unit configured to detect pulses and output a first pulse to perform a first treatment;
a second unit configured to output a second pulse to perform a second treatment; and
a control unit electrically connected to the first unit and the second unit; and
a non-transitory machine-readable medium having instructions stored thereon which, when executed by the control unit, configures the control unit to:
monitor pulses and activities of the first unit and the second unit; and
prevent the first unit from mistakenly outputting the first pulses or failing to output the first pulses when a first body part of the subject needs the first pulses for the first treatment.

20. The implantable medical device of claim 19, wherein the control unit includes:
a processor; and
a monitor unit connected to the processor, the monitor unit being configured to monitor pulses and generate monitor results based on the pulses detected, wherein the pulses includes the second pulse from the second unit; wherein
the non-transitory machine-readable medium further includes instructions stored thereon which, when executed by the processor, configure the processor to:
analyzes the monitor results;
determines whether the detected pulses originates from the second unit or from the first body part in need of the first pulses for the first treatment;
determines whether the pulses detected originates from the second unit or from the functioning first body part; and
notify the first unit of the determination by the processor.
